(19) United States
(12) Patent Application Publication
(10) Pub. No.: US 2026/0207963 A1
(43) Pub. Date: Jul. 23, 2026

(54) VARIABLE COLOR LIGHTING SYSTEM AND METHOD

(71) Applicant: KORRUS, INC., Los Angeles, CA (US)

(72) Inventor: Benjamin Harrison, Los Angeles, CA (US)

(21) Appl. No.: 19/568,473

(22) Filed: Mar. 16, 2026

Related U.S. Application Data
(63) Continuation of application No. PCT/US25/46606, filed on Sep. 16, 2025.
(60) Provisional application No. 63/695,108, filed on Sep. 16, 2024.

(51) Int. Cl. A61N 5/06; H05B 47/105

(52) U.S. Cl. CPC A61N 5/0613; H05B 47/105; A61N 2005/0626; A61N 2005/0659

(57) ABSTRACT

A method of operating a wellness optimization system, comprising: sensing a state of a subject; sensing a state of an environment at the subject; determining a target physiologic, cognitive, or circadian state for the subject; controlling a multichannel lighting system to emit a light output based on said target state; re-sensing the state of the subject or the environment; and adjusting said light output in closed loop to drive the subject toward said target state.

### REFERENCE TO RELATED APPLICATIONS

This application is a continuation in part of and claims priority to PCT Application No. PCT/US2025/046606 filed Sep. 16, 2025, which is based on U.S. Provisional Application No. 63/695,108, filed Sep. 16, 2024 and Provisional Application No. 63/862,205, filed Aug. 18, 2025, all of which are incorporated herein by reference in their entirety.

### FIELD OF INVENTION

The present invention pertains generally to lighting systems utilizing light-emitting diodes (LEDs), and more specifically to systems and methods for achieving high color rendering index (CRI) and metameric stability in white light generation using commercially available LEDs, while managing blue spectral content.

### BACKGROUND OF INVENTION

In the field of LED-based lighting, achieving desirable color properties such as high CRI and consistent color appearance often requires precise control over the spectral power distribution (SPD) of the light output. Conventional approaches frequently rely on proprietary LED components tailored to specific color science (CS) parameters. For instance, early designs in this domain have utilized dedicated high-CS blue LEDs and low-CS blue LEDs-proprietary devices engineered to provide distinct blue spectral contributions for blending into white light with targeted chromaticity and rendering qualities. Suppliers have historically provided such specialized LEDs, enabling systems that balance blue content for applications demanding high fidelity in color reproduction.

These CS-based lighting systems typically incorporated proprietary high-CS and low-CS blue LEDs to achieve a controlled blue spectral profile in the resulting white light.

While these lighting systems functioned adequately, the use of proprietary LEDs rendered the product susceptible to supply chain issues as suppliers often decide to discontinue proprietary LEDs if the volume is not sufficient. These disruptions highlight the vulnerabilities of relying on proprietary LEDs, which are often subject to limited availability, production changes, and supplier dependencies. Therefore, there is a need for lighting systems that are not based upon proprietary LEDs. The present invention fulfills the above-mentioned need, among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

Faced with the supply chain challenges described above, Applicant recognized the need to redesign to transition to commercially available LEDs to ensure supply chain reliability and scalability, while still addressing the core need to manage blue content effectively. This blue color management is important to maintain metameric properties-ensuring that the light's color appearance remains stable and consistent across different observers and viewing conditions, without perceptual shifts due to spectral mismatches.

The resulting redesigned lighting system incorporates multiple channels of commercially available LEDs. Specifically, the lighting system individually drives at least four different color channels, cyan (C), lime (L), orange (O) and violet (V), herein the “CLOV” system. Such a system not only provides versatility in managing blue light content while maintaining metameric stability, but also provides a number of unexpected results and benefits.

First, Applicant discovered unexpectedly that very high CRI could be achieved across a broad range of correlated color temperatures (CCTs) and SPDs using these commercially available LEDs. This outcome was unanticipated because prevailing assumptions in the field suggested that high CRI would necessitate wide spectral channels or complex multi-LED configurations to cover the necessary color gamut. (The LEDs used in the CLOV system have relatively narrow bandwidth compared to conventional high fidelity CS lighting systems.) Contrary to expectations, the blue content management approach enabled superior CRI performance without such elaborate setups, providing a bonus in efficiency and simplicity.

Second, the lighting system of the present invention eliminates dependence on proprietary high-and low-CS blue LEDs by leveraging off-the-shelf commercial alternatives, thus enhancing overall system robustness against supply chain disruptions, offering practical benefits for manufacturing and deployment in various lighting applications.

Third, the quality of the product is improved. Because the lighting system's output is configured by the controller, the system can tolerate variability in the light-source manufacturing. In other words, the light output is not directly dependent on the consistency of the individual light sources, since the controller can adjust the power to the different light sources to ensure a consistent output.

Fourth, the configurability of the lighting system reduces inventory needs.

Specifically, because the lighting system's output can be configured as desired, the same lighting system can be used for many different applications, thereby reducing inventory requirements.

Still other advantages and unexpected benefits will be obvious to those of skill the art in light of this disclosure.

Accordingly, in one embodiment, the present invention relates to A lighting system comprises: (a) a 1st channel comprising a 1st light emitter for emitting a 1st light having a dominant wavelength from 460 nm to 500 nm; (b) a 2nd channel comprising a 2nd light emitter for emitting a 2nd light having a dominant wavelength from 540 nm to 590 nm; (c) a 3rd channel comprising a 3rd light emitter for emitting a 3rd light having a peak wavelength from 590 to 640 nm; (d) a 4th channel comprising a 4th light emitter for emitting a 4th light having a peak wavelength from 400 to 440 nm; (e) a controller for driving said 1st, 2nd, 3rd, and 4th channels independently to cause said lighting system to emit white light comprising a combination of two or more of said 1st, 2nd, 3rd, or 4th lights.

In one embodiment, the present invention relates to a method of manufacturing different lighting systems, each system having a different fixed CCT and/or a different fixed mode, each lighting system comprising, identical multiple channels, each channel comprising a light emitter for emitting a light of a different wavelength; an identical controller for driving said multiple channels independently to cause said each system to emit white light comprising a combination of two or more of said 1st, 2nd, 3rd or 4^{th} lights in a fixed mode at a fixed CCT; and said method comprising: (a) configuring said controller to drive said 1st, 2^{nd}, 3^{rd}, and 4th channels differently for each system to cause each system to emit white light comprising a combination of two or more of said 1st, 2nd, 3^{rd}, or 4th lights having at least a different fixed mode or a different fixed CCT. Or a different CCT range.

In one embodiment, the present invention relates to a smart lighting system comprising: (a) a light source; (b) a controller operatively coupled to the light source; (c) a processor configured to determine a current operational mode selected from a plurality of predefined modes including a day mode, an evening mode, and a night mode; (d) a time-based engine configured to determine a current temporal condition based on at least one of: local time, solar time, body clock, environment time, or target wake-up time; wherein the processor is configured to: (i) automatically adjust the light source to operate in a mode appropriate for the current temporal condition; wherein the processor is optionally configured to (ii) detect a user request to change the operational mode; (iii) determine whether the requested mode aligns with the current temporal condition; and (iv) when the requested mode does not align, generate a warning to the user indicating the misalignment.

In one embodiment, the present invention relates to a smart lighting system for promoting circadian alignment, comprising: (a) at least one light fixture having a controllable light source; (b) a controller operatively coupled to the light source; (c) a plurality of sensors configured to measure environmental light exposure, user sleep data, user activity data, and biological parameters; (d) a non-transitory memory storing instructions; and (e) a processor in communication with the controller and the sensors, the processor configured to: (i) classify a current temporal condition based on sensor data and a machine-learned circadian model trained to infer body time and environment time; (ii) select a lighting mode from a plurality of modes comprising a day mode, an evening mode, and a night mode, based on the classified temporal condition; (iii) generate a personalized lighting schedule aligned with a user-specified target wake time; (iv) modify the light source state in accordance with the selected lighting mode, including when the light source transitions from an off state to an on state; (v) receive a user request to override the lighting mode; (vi) determine a degree of misalignment between the user-requested mode and the classified temporal condition; and (vii) in response to a threshold misalignment, provide a warning notification to the user.

### BRIEF DESCRIPTION OF FIGURES

FIG. 1. shows the SPDs of the four channels of one embodiment of the present invention.

FIG. 2 illustrates the small shift in CCT between the MaxBlue and ZeroBlue modes of one embodiment of the present invention.

FIG. 3 shows the high CRI ridge through a CCT range in the fidelity mode of one embodiment of the present invention.

FIG. 4 shows the relative channel strength ratios for the MaxBlue and ZeroBlue modes of one embodiment of the present invention.

FIG. 5 shows one embodiment of the operator interface on a smart phone.

FIG. 6 is a schematic diagram of one embodiment of the present invention.

### DETAILED DESCRIPTION

Throughout this description, the preferred embodiment and examples shown should be considered as examples, rather than as limitations on the present invention. This invention may, however, be embodied in many different forms and should not be construed as limited to the embodiments set forth herein; rather, these embodiments are provided so that this disclosure will be thorough and complete, and will fully convey the scope of the invention to those skilled in the art. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

The CLOV system is a multi-channel LED lighting architecture designed to deliver customizable light outputs across a range of correlated color temperatures (CCTs), typically from about 2000K to 6500K or higher, with selectable modes that prioritize different performance criteria such as circadian stimulation, color fidelity, energy efficiency, brightness, and specialized functions like antibacterial effects or near-infrared (NIR) pulsing. In various embodiments, the system comprises four primary LED channels, which may be supplemented by additional channels (e.g., a fifth NIR channel) for enhanced functionality. The channels are selected from commercially available LEDs (such LEDs are commercially available from, for example, Lumileds and Cree), enabling scalability, cost-effectiveness, and supply chain reliability.

The system achieves its versatility by dynamically adjusting the relative intensities and ratios of the LED channels, particularly by balancing the usage of violet and cyan (or blue-equivalent) channels with a lime channel. In one embodiment, this balancing allows the system to transition between modes while minimizing perceptual color shifts, ensuring that the light appears consistent to the human eye under varying conditions.

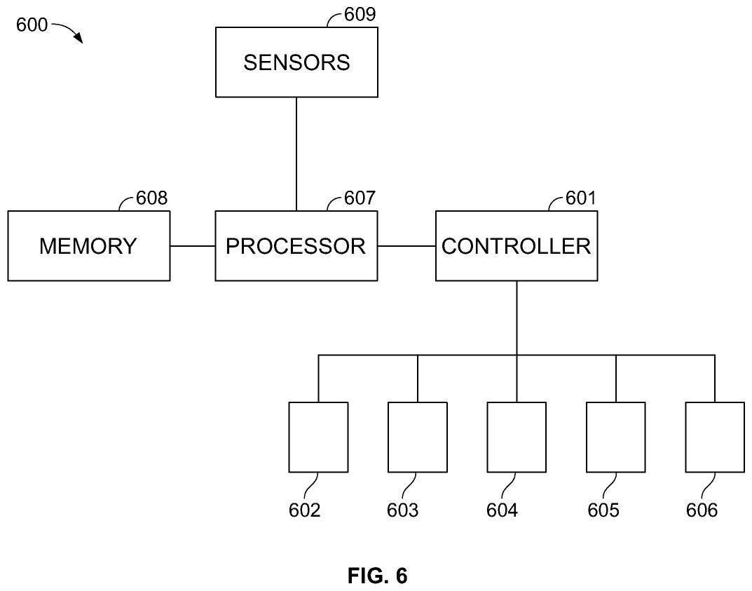

Referring to FIG. 6, one embodiment of the lighting system 600 of the present invention is shown. The system 600 comprises: a 1st channel 602 comprising a 1st light emitter for emitting a 1st light having a dominant wavelength from 460 nm to 500 nm; a 2nd channel 603 comprising a 2nd light emitter for emitting a 2nd light having a dominant wavelength from 540 nm to 590 nm; a 3rd channel 604 comprising a 3rd light emitter for emitting a 3rd light having a peak wavelength from 590 to 640 nm; a 4th channel 605 comprising a 4th light emitter for emitting a 4th light having a peak wavelength from 400 to 440 nm; a controller 601 for driving said 1st, 2nd, 3rd, and 4th channels independently to cause said lighting system to emit white light comprising a combination of two or more of said 1st, 2nd, 3rd, or 4th lights. These features are described in greater detail and with respect to selected alternative embodiments below.

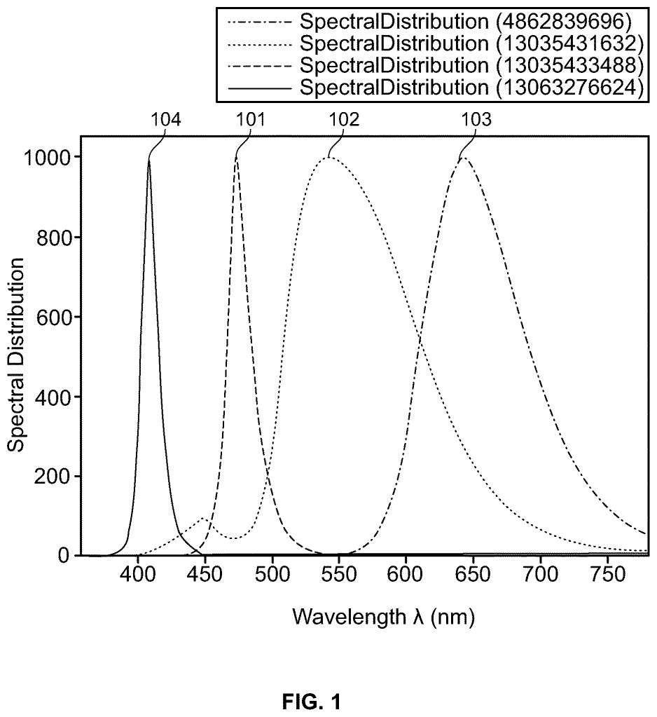

The CLOV system employs readily available LED channels from multiple manufacturers, available in various packages suitable for directional (e.g., spotlights) and non-directional (e.g., omnidirectional bulbs) lighting applications. These channels exhibit specific SPDs (spectral power distributions), which, in one embodiment, may be characterized as follows and referring to FIG. 1 for illustrative SPD plots:
Channel 1 101: Cyan (or Blue-equivalent): Provides a cyan-blue spectral peak for blue content control. In one embodiment, peak emission around 480-500 nm, contributing to blue/cyan energy.
Channel 2 102 Lime: Offers a broad green-yellow spectrum for efficiency and color balancing. In one embodiment, it has broad emission from 500-600 nm, with a peak in green-yellow for high efficacy.
Channel 3 103: PC Red-Orange: Contributes red-orange wavelengths for warmth and color gamut extension. In one embodiment, it has a peak around 590-620 nm, enhancing red rendering.
Channel 4 104: Violet: Enables violet spectral contributions for metameric adjustments and specialized modes. In one embodiment, it has a peak around 400-420 nm, allowing fine-tuned short-wavelength control.

In some embodiments, a fifth channel 606 (FIG. 6), such as Near IR (e.g., 700-850 nm) is added. This enables advanced features like photobiomodulation, brainwave entrainment, and circadian modulation without visible disruption. In one embodiment, a lower wavelength violet (e.g., 380-410) is used in a 5^{th} channel to provide warm dim/circadian/antibacterial modes.

The CLOV system has a driver that dynamically adjusts channel ratios to achieve the desired modes, often maintaining metameric equivalence for seamless transitions. In one embodiment, the driver is a commercially available RGBWW (Red-Green-Blue-White (cool)-White (Warm) platform. Of primary interest are the following operational modes:

MaxBlue Mode: Optimized for delivering maximum circadian stimulation during biological daytime hours, emphasizing higher blue spectral content. In one embodiment, in MaxBlue mode, the fourth channel is not driven.

ZeroBlue Mode: Designed to minimize or avoid circadian stimulation during biological nighttime, reducing blue spectral content to near zero while preserving white light appearance. In one embodiment, in the zero blue mode, the first channel is not driven.

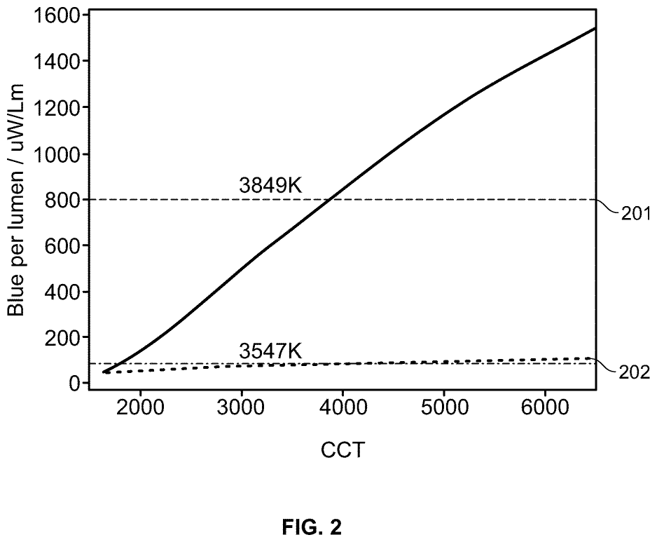

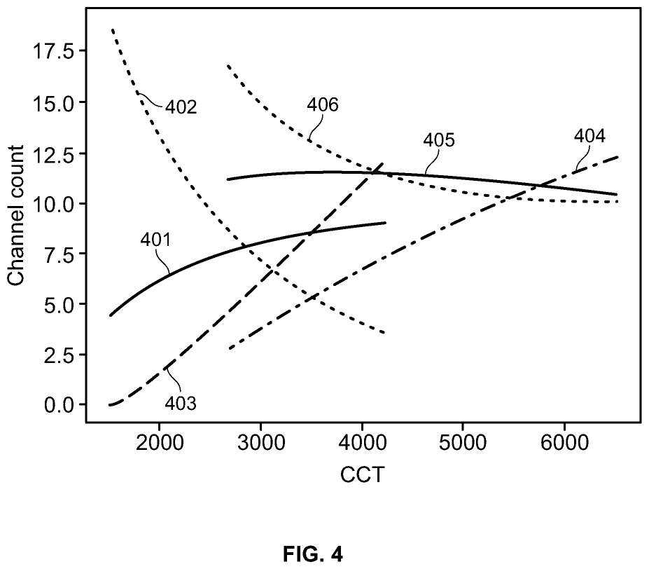

In one embodiment, the system's channels enable metameric light generation, where different spectral power distributions (SPDs) produce visually identical colors. For example, referring to FIG. 2, by adjusting violet and cyan/blue channels, the system can operate in high-blue (MaxBlue) or low-blue (ZeroBlue) modes 201, 202 with minimal CCT shift (e.g., from 3547K in low-blue to 3849K in high-blue, a difference of approximately 300K), while maintaining consistent chromatic appearance. To this end, referring to FIG. 4, the three lines 401, 402, 403 that start on the left (2^{nd}, 3^{rd}, and 4^{th} channels, respectively) show the channel ratios for ZeroBlue operation, and the three lines 404, 405, 406 on the right (1^{st}, 2^{nd}, and 3^{rd} channels, respectively) show the ratios for MaxBlue.

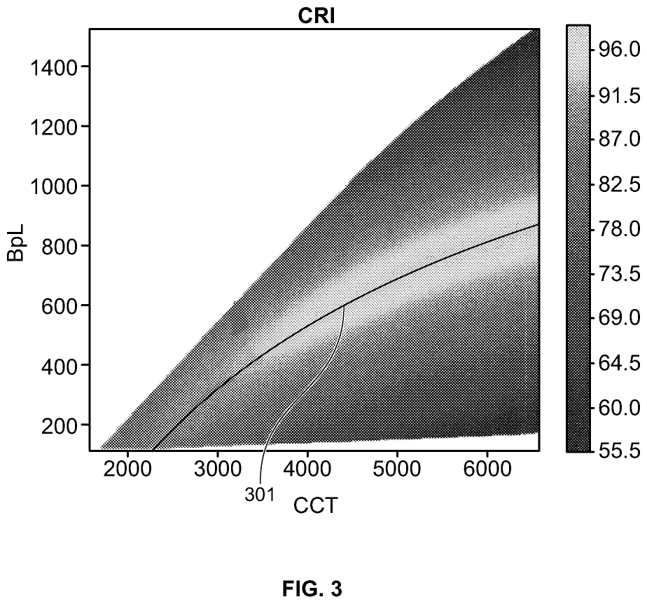

Fidelity Mode: Focused on delivering high-quality light as measured by metrics such as CRI, IES TM-30, or other color fidelity indices, often achieving CRI values of 90 or above. In one embodiment, the violet and cyan channels are balanced in a ratio of approximately 40-60% (e.g., 50/50), with the lime channel providing a complementary broad-spectrum contribution to maintain metameric stability and high CRI by following a “yellow ridge” 391 through the center of a mode triangle in a CRI-vs-CCT plot (See FIG. 3) in achieving for example a high CRI, such as 96.

Efficacy Mode: Prioritizes maximizing lumens per watt (1 m/W) while maintaining a minimum acceptable CRI (e.g., 84 or higher). In one embodiment, just the more efficient 1^{st}, 2^{nd}, and 3^{rd} channels are driven.

Brightness Mode: Maximizes total light output within system constraints, potentially incorporating driver adjustments and compensation strategies. Specifically, brightness mode maximizes luminous flux given driver constraints. As intensity increases, one channel (e.g., cyan/blue) may reach its maximum current limit. The system then compensates by increasing others (e.g., lime and violet) until their limits are reached. In a “disaster mode” embodiment, users can flick the light switch multiple times to temporarily boost brightness, and, in one embodiment, this overrides limits for emergency use.

Warm Dim, Circadian, and Antibacterial Modes: These mode uses a 5^{th} channel with a shorter wavelength violet light to provide dimming with warming CCT shifts (e.g., from 3000K to 1800K), circadian-aligned scheduling, and/or antibacterial effects.

Circadian Modulation Mode: This mode uses a light source to emit tailored spectral light pulses (or high-frequency flicker) that alter melanopsin stimulation without changing apparent brightness or color. In one embodiment, this is achieved through silent-substitution of metamers using the multi-channel LED engine. The fixture is calibrated with at least two spectral states: State A and State B, which produce the same photopic luminance (Y) and nearly the same color coordinates (u′, v′)—so that to the human eye they appear steady and unchanging—yet one state has higher melanopic content (more stimulation of ipRGCs) than the other. By rapidly alternating between these states, the system can introduce a periodic melanopic modulation. For example, State A might be a white light spectrum rich in long-wavelength content (low melanopic lux) and State B a different mix with more 480 nm content (high melanopic lux), but both are tuned to appear as the same neutral white to observers. By flickering between A and B, the ipRGCs in the eye sense a flicker (because melanopsin responds differently) while the L/M/S cones see no flicker. The modulation can be delivered as continuous high-frequency flicker (e.g. 100 Hz alternating A/B, which is beyond the fusion threshold for cone vision) or as discrete pulse trains (e.g. brief pulses of the melanopically enriched state at strategic times). The temporal strategy can be adjusted based on chronobiological goals: for instance, in the morning, a higher melanopic pulse frequency or duty cycle may be used to boost alertness, whereas in the evening, the system might either refrain from melanopic stimulation or even do the opposite (e.g. a “dark metamer” pulse that selectively stimulates rods/cones without melanopsin to pre-adapt the circadian system to darkness). Importantly, the photometric constraints are tightly controlled—the difference in luminance (ΔY) between states is minimal (typically near 0), and the chromaticity shift (Δu′v′) is kept below just-noticeable thresholds (on the order of 0.001-0.004). Likewise, the relative stimulation of L, M, S cones is held constant (LMS “silent”), so the flicker does not induce color or brightness artifacts. This requires careful spectral optimization using 5 or more LED primaries and feedback calibration. The system's firmware can compute the required drive currents for each channel to achieve the two metameric spectra, given the spectral power distributions of the LEDs and the target melanopic contrast.

During operation, a high-speed driver or PWM mechanism alternates the LED outputs between these calibrated settings. Because multiple channels are involved, the transitions are synchronized and smoothed to avoid any electrical or optical transients. The result is a circadian lighting input that can be delivered unobtrusively in the background of normal illumination.

Photobiomodulation Mode: This mode uses a light source in the 5^{th} channel to emit therapeutic NIR (810-830 nm) and/or deep red (~660 nm) light at controlled irradiance levels to promote mitochondrial and cellular health. The dosing (power density and energy delivered) can be adjusted and timed according to therapy protocols (e.g. 5-20 min sessions at 10-100 mW/cm²), or other protocols based on longer, ambient exposure. In one embodiment, built-in thermal management (heat sinks, temperature sensors, and drive current limits) ensures the LEDs operate within safe junction temperatures and that skin exposure remains comfortable. The system may include safety measures and interlocks compliant with photobiological safety standards (to prevent accidental overexposure to invisible NIR). For example, it may automatically dim or shut off the NIR output after a maximum exposure time or if it detects an overheating condition. User controls (or a companion app) allow selection of PBM sessions targeting various outcomes (e.g. general wellness, muscle recovery, cognitive stimulation), with presets for recommended irradiance and duration.

Brainwave Entrainment Mode: This mode uses a NIR light source in the 5^{th} channel to emit in a pulsed pattern at specific brainwave frequencies to induce neural entrainment. In one embodiment, the NIR emitter (810 nm) is pulsed in a square-wave or duty-cycled manner at frequencies such as ~10 Hz (alpha rhythm), ~40 Hz (gamma rhythm), or other frequencies of interest (in the range of about 1 Hz up to ~100 Hz). Because 810 nm light is invisible, users can experience the neural effects (via transcranial penetration of the NIR) without perceiving any flicker. The duty cycle and amplitude of pulses are programmable-for instance, a 40 Hz pulse train at 50% duty cycle has been shown effective for increasing gamma-band activity in the brain. The system can thus provide cognitive and neurological stimulation integrated into a normal-looking lamp. In general-purpose illumination mode, the brainwave pulsing may be disabled or kept at imperceptibly high frequencies; it is activated intentionally for therapy sessions. The device may guide the user through safe session lengths (e.g. 20 minutes of 40 Hz stimulation, with automatic shutoff) and provide warnings or lockouts if a potentially sensitive individual (e.g. someone with a history of epilepsy) tries to enable a visible flicker frequency. In specialized implementations (such as a therapeutic lamp or wearable headset), visible-light entrainment is also possible—e.g. pulsing a dim visible red light at 10 Hz for closed-eye relaxation training—but the primary approach is to use NIR for invisible entrainment to avoid disturbing the user's vision.

The circadian modulation, photobiomodulation, and brainwave entrainment modes are health-related modes which are not isolated-they can operate in combination within the same lighting device or system. For example, a ceiling light could concurrently provide a 40 Hz NIR pulsing (for brain stimulation) while also running a silent melanopsin-modulating flicker at a higher frequency on the visible channels to enhance alertness. Meanwhile, that same device could schedule a separate PBM session (e.g. an hour before bed, it could switch to an 810 nm therapy mode at low visible brightness for relaxation/recovery). The invention's multi-channel design and software control allow these features to be used independently or simultaneously, as appropriate. The system is extensible: as LED technology advances, additional channels (6, 7, or more different wavelengths) can be incorporated to further refine the spectral control or add new therapy modalities. The architecture is prepared to handle such expansion-for instance, by abstracting each channel's contribution and using a matrix-based color mixing approach, adding a new LED (say, an amber or a violet) simply provides another degree of freedom for spectral tuning. Similarly, the control firmware can accommodate new waveform protocols (for brain or circadian stimulation) as research develops, by updating the driving software without changing hardware.

In summary, the health-related modes transforms the lighting system into a versatile health-support device. It maintains full functionality as general illumination (high-quality white light output, dimmable, color-tunable) and adds layered therapeutic capabilities: invisible NIR therapy for body and brain, embedded neurological rhythm support, and circadian rhythm optimization through spectral engineering. All of these are achieved within a single lighting platform that can take the form of retro-fit bulbs, fixtures, or integrated architectural lighting installations.

In one embodiment, the system includes a user interface (UI) for mode selection and monitoring, accessible via a smart phone application, wall switch, or integrated display.

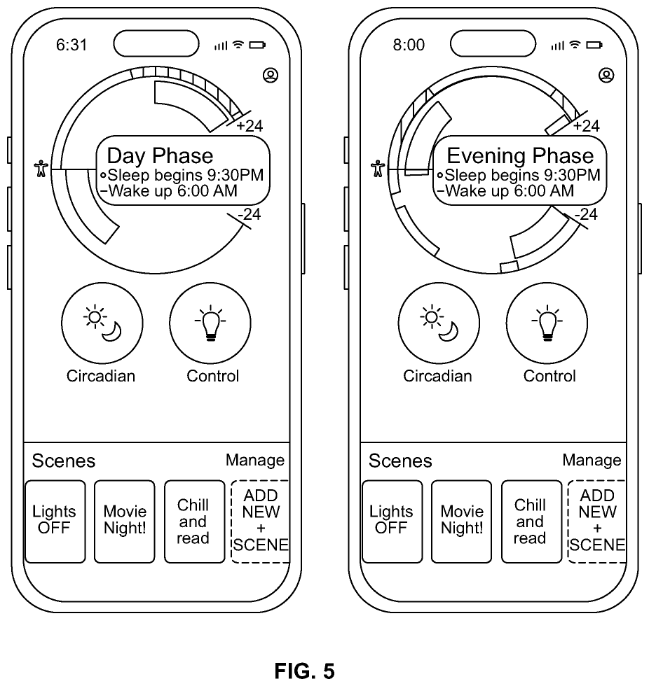

Referring to FIG. 5, a smart phone UI application is shown.

In one embodiment, the lights of the system will automatically change throughout the day, including when they are off. This means that if the user turns on a light it will come in in the correct mode, rather than simply resuming whatever it was doing when it was turned off. In the simplest case, this is controlled by clock time, but in a more sophisticated scenario, the ‘time’ might be based on someone's body clock or some target outcome.

In one embodiment, the lights have distinct modes of operation (day/evening/night). If a user attempts to use a mode at the wrong time, then the system will alert them that they are doing so. For example, if it is evening and they ask for something in the day category, then the system will warn them that it is not a good idea.

In one embodiment, the system will be producing a schedule based on the user's target wake up time.

Multi-Clock Representation: The UI displays multiple “clocks” including local time, solar time, body clock (derived from sleep/activity data), environment time (based on recent light exposure), and target time. Greater alignment among these indicates better circadian health. In one embodiment, the partial ring at the top represents 24 hours into the future and 24 into the past as shown in FIG. 5.

In one embodiment, the system may be integrated with sensors that measure light exposure, sleep patterns, activity levels, and/or biological parameters (e.g., via wearables), just to name few. Data is presented in the UI and used to compute derived times (e.g., body or environment time), enabling adaptive scheduling.

The embodiments described herein are illustrative, and variations (e.g., additional channels, alternative part numbers, or UI customizations) are within the scope of the invention.

In one embodiment, the present invention relates to a lighting system comprises: (a) a 1st channel comprising a 1st light emitter for emitting a 1st light having a dominant wavelength from 460 nm to 500 nm; (b) a 2nd channel comprising a 2nd light emitter for emitting a 2nd light having a dominant wavelength from 540 nm to 590 nm; (c) a 3rd channel comprising a 3rd light emitter for emitting a 3rd light having a peak wavelength from 590 to 640 nm; (d) 4th channel comprising a 4th light emitter for emitting a 4th light having a peak wavelength from 400 to 440 nm; and a controller for driving the 1st, 2nd, 3rd, and 4th channels independently to cause the lighting system to emit white light comprising a combination of two or more of the 1st, 2nd, 3rd, or 4th lights.

In one embodiment, the controller is configured to control two or more of the 1st, 2nd, 3rd, and 4th channels independently to emit the white light in a desired mode. In one embodiment, the controller drives three or more of the 4th, 1st, 2nd, and 3rd channels to emit the white light in the desired mode.

In one embodiment, the desired mode is fixed by the controller. In one embodiment, the desired mode is variable by driving the 1st, 2nd, 3rd, and 4th channels differently. In one embodiment, the desired mode is at least one of the following modes, a high-blue mode, a low-blue mode, and high-fidelity mode, a high efficiency mode, a high-brightness mode, or a minimum quality, efficiency mode. In one embodiment, the high-blue mode, the 4th channel is not driven; in the low-blue mode, the 1st channel is not driven; in the high-fidelity mode, all channels are driven; in the high efficiency mode the 1st and 2nd channels are preferentially driven over the 3rd channel and the 4th channel is not driven; and in the high-brightness mode, all channels are driven; in the minimum quality, efficiency mode, the use of the 4th channel is minimized subject to meeting the minimum quality requirement.

In one embodiment, the health-related mode is a circadian modulation mode in which tailored spectral light pulses are emitted that alter melanopsin stimulation without changing apparent brightness or color. In one embodiment, the pulses are metamers using two different configures of the channels or possibly a 5th channel, in which the two different configurations produce first and second light having essentially the same photopic luminance and nearly the same color coordinates, so that to the human eye they appear steady and unchanging yet the first light has higher melanopic content than said second light.

In one embodiment, the health-related mode is a photobiomodulation mode in which a 5th channel emits therapeutic NIR (810-830 nm) and/or deep red (~660 nm) light at controlled irradiance levels to promote mitochondrial and cellular health.

In one embodiment, the health-related mode is a brainwave entrainment mode in which the 5th channel comprises a NIR light source to emit in a pulsed pattern at specific brainwave frequencies to induce neural entrainment. In one embodiment, the NIR emitter (810 nm) is pulsed in a square-wave or duty-cycled manner at frequencies such as ~10 Hz (alpha rhythm), ~40 Hz (gamma rhythm), or other frequencies of interest (in the range of about 1 Hz up to ~100 Hz).

In one embodiment, the controller drives three of more of the 4th, 1st, 2nd, and 3rd channels to emit the white light at a desired CCT. In one embodiment, the desired CCT is variable by driving the 1st, 2nd, 3rd, and 4th channels differently. In one embodiment, the desired CCT is a fixed CCT in the controller. In one embodiment, the controller is configured to be reset to drive three of more of the 4th, 1st, 2nd, and 3rd channels differently to emit the white light at a 1st fixed CCT different form the fixed CCT.

In one embodiment, the 1st light emitter is saturated. In one embodiment, the 2nd light emitter is unsaturated. In one embodiment, the 3rd light emitter is unsaturated. In one embodiment, the 4th light emitter is saturated In one embodiment, the 1st, 2nd, 3rd, and 4th light emitters are LEDS. In one embodiment, the 1st, 2nd, 3rd, and 4th light emitters are OLEDS.

In one embodiment, the lighting system is a lamp. In one embodiment, the lighting system is a panel. In one embodiment, the lighting system is an integrated fixture. In one embodiment, the lighting system is a pixelated display and the 1st, 2nd, 3rd, and 4th light emitters are subpixels. In one embodiment, the subpixels are microLEDS. In one embodiment, the subpixels are OLEDS. In one embodiment, the lighting system is a backlight.

In one embodiment, the 1st light has a peak wavelength from 470 nm to 490. In one embodiment, the 1st light has a peak wavelength of 480 nm. In one embodiment, the 2nd light has a peak wavelength from 550 nm to 580 nm. In one embodiment, the 2nd light has a peak wavelength of 558 nm. In one embodiment, the 3rd light has a peak wavelength from 610 to 630 nm. In one embodiment, the 3rd light has a peak wavelength from 620 nm. In one embodiment, the 4th light has a peak wavelength from 410 to 430 nm. In one embodiment, the 4th light has a peak wavelength of 420 nm.

The present invention also relates to a method of manufacturing different lighting systems, in which each system has a different fixed CCT and/or a different fixed mode, each lighting system comprising: identical multiple channels, each channel comprising a light emitter for emitting a light of a different wavelength; an identical controller for driving the multiple channels independently to cause the each system to emit white light comprising a combination of two or more of the 1st, 2nd, 3rd, or 4th lights in a fixed mode and/or at a fixed CCT. In one embodiment, the method comprises: configuring the controller to drive the 1st, 2nd, 3rd, and 4th channels differently for each system to cause each system to emit white light comprising a combination of two or more of the 1st, 2nd, 3rd, or 4th lights having at least a different fixed mode or a different fixed CCT or a different CCT range.

In one embodiment, the fixed mode comprises at least one of the following modes, a high-blue mode, a low-blue mode, and high-fidelity mode, a high efficiency mode, or a high-brightness mode, or a health related mode. In one embodiment, the identical multiple channels are the same channels as described above in connection with the lighting system.

In one embodiment, the method further comprises resetting the controller of one of the different lighting systems to drive the 1st, 2nd, 3rd, and 4th channels independently to emit the white light in at least a different 1st fixed mode or a different 1st fixed CCT. In one embodiment, the method comprises resetting the controller of one of the different lighting systems to drive the multiple channels differently to emit the white light in at least a different 1st fixed mode or a different 1st fixed CCT.

In one embodiment, the present invention relates to a smart lighting system comprising: (a) a light source; (b) a controller operatively coupled to the light source; (c) a processor configured to determine a current operational mode selected from a plurality of predefined modes including a day mode, an evening mode, and a night mode; (d) a time-based engine configured to determine a current temporal condition based on at least one of: local time, solar time, body clock, environment time, or target wake-up time; wherein the processor is configured to: (i) automatically adjust the light source to operate in a mode appropriate for the current temporal condition; wherein the processor is optionally configured to (ii) (b) detect a user request to change the operational mode; (iii) determine whether the requested mode aligns with the current temporal condition; and (iv) when the requested mode does not align, generate a warning to the user indicating the misalignment.

In one embodiment, one or more sensors configured to measure one or more of: ambient light exposure, user sleep duration, physical activity, cycles of activity and rest, or biological parameters. In one embodiment, at least one wearable sensor configured to collect at least one of light exposure, sleep stages, or actigraphy data in real-time. In one embodiment, data from the sensors are used to compute a dynamic environment time indicative of the effective circadian phase experienced by the user.

In one embodiment, the controller adjusts the light source's state even when the light source is off, such that upon activation the light source operates in a mode aligned with the current temporal condition. In one embodiment, the temporal condition is determined based on a personalized circadian schedule inferred from sensor data.

In one embodiment, the system further comprises a scheduling module configured to generate a personalized lighting schedule based on a user's target wake-up time.

In one embodiment, a user interface is configured to simultaneously display multiple clocks, including two or more of local time, solar time, body clock time, environment time, and target wakeup time.

In one embodiment, the system is configured to promote greater coherence between these clocks which results in better circadian health. In one embodiment, the interface indicates a circadian coherence score representing alignment among the time representations.

In one embodiment, the user interface comprises a ring visual element representing a 48-hour period, segmented into past and future intervals.

In one embodiment, the light fixture includes a multichannel LED array capable of emitting variable spectral profiles corresponding to melanopic sensitivity curves.

In one embodiment, the controller comprises a microcontroller with a real-time clock and is powered by a low-voltage DC circuit integrated into a smart home hub.

Referring to FIG. 6, the present invention also relates to a smart lighting system for promoting circadian alignment. In one embodiment, the system 600 comprises: (a) at least one light fixture having at least one controllable light source 602-606; (b) at least a controller 601operatively coupled to the at least one light source; (c) a plurality of sensors 609 configured to measure environmental light exposure, user sleep data, user activity data, and biological parameters; (d) a non-transitory memory 608 storing instructions; and (e) a processor 607 in communication with the controller and the sensors, the processor configured to: (i) classify a current temporal condition based on sensor data and a machine-learned circadian model trained to infer body time and environment time; (ii) select a lighting mode from a plurality of modes comprising a day mode, an evening mode, and a night mode, based on the classified temporal condition; (iii) generate a personalized lighting schedule aligned with a user-specified target wake time; (iv) modify the light source state in accordance with the selected lighting mode, including when the light source transitions from an off state to an on state; (v) receive a user request to override the lighting mode; (vi) determine a degree of misalignment between the user-requested mode and the classified temporal condition; and (vii) in response to a threshold misalignment, provide a warning notification to the user.

In one embodiment, the processor is further configured to continuously retrain the machine-learned circadian model based on feedback from new sensor data collected over time.

In one embodiment, the environment time is computed by evaluating recent light exposure patterns and activity rhythms using a neural network model.

In one embodiment, the body time is derived using a fusion model incorporating heart rate variability, motion data, and ambient light cues.

In one embodiment, the controller modifies one or more of: color temperature, light intensity, and spectral composition of the light source based on the selected lighting mode.

In one embodiment, the warning notification comprises a visual display in the user interface and optionally includes a recommendation for a more aligned mode.

In one embodiment, override requests are logged and used to adapt the personalized lighting schedule via reinforcement learning.

In one embodiment, a graphical user interface configured to display multiple synchronized time representations including at least two or more of local time, solar time, body time, environment time, and target circadian alignment time; and displays a coherence score representing the degree of alignment among the multiple time representations.

In one embodiment, the interface includes a circular timeline comprising a 24-hour past and 24-hour future representation, with color-coded segments denoting recommended light exposure windows.

In additional embodiments, the present invention relates to a wellness-optimization system that operates in closed loop to improve the health and wellness of a subject. The subject may be a human or a non-human animal. The system monitors a state of the subject, monitors a state of the environment at the subject, monitors one or more controllable environmental variables, determines a desired physiologic, cognitive, or circadian state for the subject, and controls one or more outputs to drive the subject toward the desired state. In one embodiment, the lighting system described herein forms the primary actuator, while in other embodiments the lighting system is coordinated with other environmental actuators.

In one embodiment, the state of the subject comprises one or more of sleep state, wake state, activity level, motion, posture, heart rate, heart rate variability, respiration, body temperature, skin temperature, blood oxygen, cognitive state, stress level, eye state, blink rate, EEG-derived information, or combinations thereof. In one embodiment, the state of the environment at the subject comprises one or more of ambient light level, spectral content, melanopic content, time of day, solar time, local time, room temperature, humidity, air quality, volatile compounds, carbon dioxide, sound level, intermittent noise, smell, occupancy, or device usage. In one embodiment, the controllable environmental variables comprise one or more of visible light intensity, visible light spectrum, correlated color temperature, melanopic content, metameric content, pulse frequency, pulse duty cycle, pulse amplitude, NIR output, temperature, airflow, humidity, white noise, sound masking, air purification, scent delivery, shade position, or combinations thereof.

In one embodiment, the processor classifies the subject as being in or approaching a state associated with sleep onset, non-REM sleep, REM sleep, relaxed wakefulness, concentration, problem solving, alertness, wake initiation, or an undesirable stress-associated state, and selects a corresponding control protocol. In one embodiment, the system repeatedly senses the subject and/or the environment and updates the protocol until a target state is reached, thereby providing closed-loop optimization rather than a fixed open-loop program.

In one embodiment, the system comprises a brain-state modulation mode in which the controller emits pulsed light selected to bias the subject toward a desired physiologic or cognitive state. In one embodiment, lower pulse frequencies are associated with sleep-support protocols, slightly higher frequencies are associated with REM-support protocols, frequencies in an alpha-associated range, for example about 8 Hz to about 12 Hz, are associated with relaxed but conscious states, frequencies in a beta-associated range, for example about 13 Hz to about 30 Hz, are associated with concentration and problem solving, and still higher frequencies are associated with alerting or wake-initiation protocols. These associations are exemplary only, and the system may select other frequencies, waveforms, duty cycles, amplitudes, or session durations based on research results, user response, or safety limits.

In one embodiment, the pulsed light used for brain-state modulation is emitted by a near-infrared channel. In one embodiment, the near-infrared channel emits in a range from about 700 nm to about 1000 nm, and preferably about 810 nm to about 830 nm. Because near-infrared light may be substantially invisible to the user and may penetrate tissue more readily than visible light, the system can deliver pulsed protocols without creating bothersome visible flicker. In one embodiment, the NIR pulsing is superimposed on ordinary illumination produced by the visible channels.

In another embodiment, the pulsed light used for brain-state modulation and/or circadian modulation is produced by alternating between first and second metameric visible-light states generated by the multi-channel light engine. The first and second states may be substantially matched in perceived color and luminance, while differing in melanopic content or in stimulation of selected photoreceptor pathways. Accordingly, the user may not consciously perceive a substantial change in light output while the system applies the modulation. In one embodiment, the metameric pulsing is used to place the subject in a concentration mode or other target state without visually distracting the subject.

In one embodiment, the brain-state modulation mode operates synergistically with the circadian modes described herein. For example, the controller may first reduce blue spectral content, place the visible output in an evening or low-blue mode, and then superimpose NIR pulsing and/or substantially imperceptible metameric pulsing selected to shift, reinforce, or reset the subject's circadian rhythm. In another embodiment, a morning protocol uses a different pulse frequency, duty cycle, and/or melanopic contrast to promote wakefulness or awareness. In still another embodiment, the system suppresses or terminates a selected pulsing protocol if sensed data indicate that the subject is overshooting the target state, undershooting the target state, or entering an undesirable state.

In one embodiment, the wellness-optimization system is integrated with additional environmental controllers. For example, the processor may coordinate the lighting system with one or more thermostats, HVAC systems, fans, humidifiers, air purifiers, carbon-dioxide reduction systems, speakers, white-noise generators, televisions, shading systems, and/or scent diffusers to reduce sleep disruption, improve concentration, or enhance recovery. In one embodiment, intermittent sounds are masked with white noise or other sound control. In one embodiment, thermal conditions and air quality are adjusted in coordination with the light protocol to improve wellness outcomes.

In one embodiment, the user interface displays a selected brain-state or wellness mode, a recommended protocol, and/or a warning when a requested protocol is inconsistent with the current subject state, current temporal condition, or a safety setting. In one embodiment, protocol selection is personalized based on historical responses of the subject, including differences between weekday and weekend behavior, social jetlag, and target wake time. In one embodiment, the system continues to update the selected wellness protocol even when visible light output is off, such that subsequent activation resumes in a protocol appropriate to the then-current subject state and temporal condition.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What it claimed is:

1-64. (canceled)

65. A smart lighting system for promoting circadian alignment, comprising:
at least one light fixture having a controllable light source;
a controller operatively coupled to the light source;
a plurality of sensors configured to measure environmental light exposure, user sleep data, user activity data, and biological parameters;
a non-transitory memory storing instructions; and
a processor in communication with the controller and the sensors, the processor configured to:
(i) classify a current temporal condition based on sensor data and a machine-learned circadian model trained to infer body time and environment time;
(ii) select a lighting mode from a plurality of modes comprising a day mode, an evening mode, and a night mode, based on the classified temporal condition;
(iii) generate a personalized lighting schedule aligned with a user-specified target wake time;
(iv) modify the light source state in accordance with the selected lighting mode, including when the light source transitions from an off state to an on state;
(v) receive a user request to override the lighting mode;
(vi) determine a degree of misalignment between the user-requested mode and the classified temporal condition; and
(vii) in response to a threshold misalignment, provide a warning notification to the user;
wherein said processor is further configured to determine a target physiologic, cognitive, or circadian state of a subject and to control said light fixture in closed loop based on a sensed state of the subject and a sensed state of an environment at the subject.

66. The system of claim 65, wherein said subject is a human or a non-human animal.

67. The system of claim 65, further comprising one or more sensors configured to measure one or more of sleep state, motion, heart rate, heart rate variability, respiration, body temperature, EEG-derived information, ambient light, temperature, humidity, air quality, carbon dioxide, sound, or smell.

68. The system of claim 65, wherein said processor is configured to control one or more controllable environmental variables comprising at least one of spectral composition, intensity, melanopic content, pulse frequency, pulse duty cycle, pulse amplitude, temperature, airflow, humidity, white noise, sound masking, air purification, carbon-dioxide reduction, scent delivery, or shading.

69. The system of claim 65, wherein said processor selects a brain-state modulation mode configured to bias the subject toward at least one of sleep onset, non-REM sleep support, REM sleep support, relaxed wakefulness, concentration, problem solving, alertness, wake initiation, or reduced stress.

70. The system of claim 69, wherein said brain-state modulation mode comprises pulsing a near-infrared channel.

71. The system of claim 70, wherein said near-infrared channel emits light from about 700 nm to about 1000 nm.

72. The system of claim 71, wherein said near-infrared channel emits light at about 810 nm to about 830 nm.

73. The system of claim 69, wherein said brain-state modulation mode comprises alternating between first and second metameric visible-light states that are substantially matched in perceived color and luminance but differ in melanopic content.

74. The system of claim 73, wherein the alternating between said first and second metameric visible-light states is substantially imperceptible to the subject.

75. The system of claim 69, wherein the pulse frequency is selected from about 1 Hz to about 100 Hz.

76. The system of claim 75, wherein frequencies in an alpha-associated range are used for relaxed wakefulness, frequencies in a beta-associated range are used for concentration or problem solving, and higher frequencies are used for alerting or wake-initiation protocols.

77. The system of claim 65, wherein said processor is configured to reduce blue spectral content of said light fixture and to superimpose a pulsed light pattern to shift, reinforce, or reset a circadian rhythm of the subject.

78. The system of claim 77, wherein said pulsed light pattern is applied while visible output remains in a low-blue, evening, or night mode.

79. The system of claim 65, wherein said processor adjusts at least one of pulse frequency, duty cycle, wavelength, spectral composition, or session duration in response to sensed progress toward said target physiologic, cognitive, or circadian state.

80. A method of operating a wellness optimization system, comprising:
sensing a state of a subject;
sensing a state of an environment at the subject;
determining a target physiologic, cognitive, or circadian state for the subject;
controlling a multichannel lighting system to emit a light output based on said target state;
re-sensing the state of the subject or the environment; and
adjusting said light output in closed loop to drive the subject toward said target state.

81. The method of claim 80, wherein controlling said multichannel lighting system comprises reducing blue spectral content and superimposing pulsed near-infrared light or substantially imperceptible metameric visible-light pulses.

82. The method of claim 80, further comprising controlling at least one non-light environmental variable selected from temperature, airflow, humidity, air quality, carbon-dioxide reduction, sound masking, intermittent-noise suppression, or scent delivery in coordination with said light output.

83. The method of claim 80, wherein said target state comprises at least one of sleep onset, non-REM sleep support, REM sleep support, relaxed wakefulness, concentration, problem solving, alertness, or wake initiation.

84. A wellness optimization system comprising:
at least one controllable light fixture having a multichannel light source including at least one visible-light channel and optionally a near-infrared channel;
one or more sensors configured to monitor a state of a subject and a state of an environment at the subject;
one or more environmental controllers configured to control at least one environmental variable in addition to light; and
a processor configured to determine a desired subject state and to control said light fixture and said one or more environmental controllers in closed loop to drive the subject toward said desired subject state.

85. The system of claim 84, wherein said one or more environmental controllers comprise at least one of a thermostat, an HVAC system, a fan, a humidifier, an air purifier, a speaker, a white-noise generator, a shade control, or a scent diffuser.

86. The system of claim 84, wherein said processor selects between a visible metameric pulsing protocol and a near-infrared pulsing protocol based on at least one of time of day, the desired subject state, sensed response of the subject, or a sensitivity setting.
